(19) United States
(12) Patent Application Publication
(10) Pub. No.: US 2020/0237606 A1
(43) Pub. Date: Jul. 30, 2020

(54) Blood Flow Restriction System

(71) Applicant: Smart Tools Plus, LLC, Strongsville, OH (US)

(72) Inventor: Nicholas Colosi, Strongsville, OH (US)

(21) Appl. No.: 16/773,102

(22) Filed: Jan. 27, 2020

Related U.S. Application Data
(60) Provisional application No. 62/797,689, filed on Jan. 28, 2019.

(51) Int. Cl. A61H 9/00

(52) U.S. Cl. CPC A61H 9/0092; A61B 17/135; A61H 2201/1409

(57) ABSTRACT

A BFRT cuff and portable electric pump system and a method for exercising using said cuff. The method incudes providing a BFRT cuff and a portable electric pump system, placing the BFRT cuff on a limb of a user, connecting the BFRT cuff to the portable electric pump system, selecting a pressure value or percent of limb occlusion pressure (LOP), causing the portable electric pump system to supply air to the BFRT cuff until the LOP of said limb is obtained, terminating air flow to the BFRT cuff and then reducing the pressure in the BFRT cuff, disconnecting the BFRT cuff from the portable electric pump system, and exercising the limb of the user while the BFRT cuff is at a selected pressure or a selected percent of LOP.

The present disclosure claims priority on United States Provisional Application No. 62/797,689 filed Jan. 28, 2019, which is incorporated herein by reference.

### FIELD OF THE DISCLOSURE

This disclosure relates to a blood flow restriction system, specifically to a blood flow restriction system that can be used to set an inflatable belt system to a specific percentage of limb occlusion pressure (LOP), and more specifically to a blood flow restriction system that can be used to measure the LOP for a particular limb of a user and then set the inflatable belt system to a specific percentage of LOP as set by the user.

### BACKGROUND OF THE DISCLOSURE

Blood Flow Restriction Training (BFRT) is becoming a popular tool to improve muscle strength, size and functional aerobic capacity in shorter amounts of time with less stress on the body than typical training. Practitioners began using BFRT in the treatment and recovery from musculoskeletal injuries or disabilities. Trainers and coaches have used BFRT as an adjunct for a usual training regimen or as a tool to aid recovery. BFRT is the brief and intermittent occlusion or restriction of arterial and venous blood flow that is performed by applying a tourniquet to the upper or lower extremity. BFRT has been found to augment skeletal muscle adaptation, along with systemic whole-body changes and cardiovascular benefits while at rest, with low intensity endurance exercises or low load resistance training. BFRT has been found to be safe when applied with pressures relative to the cuff width and individual limb circumference which is obtained through measuring limb occlusion pressures via Doppler.

BFRT is done by wrapping a tourniquet around the top portion of one's upper or lower limbs. The wrapping restricts blood flow from the veins of the working muscles to the heart and limits the amount of blood flow to the limbs from the arteries. The restriction results in physiological changes that mimic changes associated with high intensity exercise. The results are gains in muscle size and strength and increases in cardiovascular function at much lower intensities than are usually required for adaptation.

Various types of BFRT cuffs have been developed as described in U.S. Pat. Nos. 6,149,618; 7,413,527; 7,455,630; 8,021,283; 8,182,403; 8,273,114; 8,328,693; 8,366,740; 8,425,426; 8,992,397; 9,301,701; and 10,245,458; US Publication Nos. US 2009/0124912; 2015/0150560; US 2016/0193491; US 2017/0112504; US 2017/0224357; 2017/0325825; and US 2018/0290005; PCT Publication Nos. WO 2016/087123; WO 2017/149690; and WO 2019/068147; and the Smart Cuff™ devices offered by Smart Tools Plus, LLC (http://www.smarttoolsplus.com/), all of which are incorporated herein by reference.

Although these prior art BFRT cuffs can be successfully used for BFRT, these prior art BFRT cuffs typically require manual inflation/deflation, use of manual pressure gauges to monitor the pressure of the cuff, and the inconvenient adjustment of the cuff pressure during training. As such, there remains a need for a BFRT cuff having improved convenience of use during BFRT.

### SUMMARY

The BFRT cuff in accordance with the present disclosure is designed to address the continued needs of individuals using BFRT. The BFRT cuff of the present disclosure includes and/or is used with one or more of the following features:
a BFRT cuff that is simple and inexpensive to manufacture;
a BFRT cuff that is comfortable for use by the user;
a BFRT cuff that is simple to use by the user;
a BFRT cuff that is easy to apply and remove by the user;
a BFRT cuff that is compact, lightweight, and easy to use, transport, and store;
a BFRT cuff that is can be used for a wide range of user body types;
a BFRT cuff that is washable;
a BFRT cuff that is durable;
a BFRT cuff that contours to the body when inflated;
a BFRT cuff that includes one or more air chambers;
a BFRT cuff that is at least 11 inches long (e.g., 11-30 inches);
a BFRT cuff that has a urethane-coated air bladder;
a BFRT cuff that includes a motor/pump unit to inflate/deflate the one or more air chambers in the BFRT cuff;
a BFRT cuff that includes a motor/pump unit to inflate/deflate the one or more air chambers during use of the BFRT cuff;
a BFRT cuff that includes a motor/pump unit that is detachable/connectable to the BFRT cuff and can remain connected to the BFRT cuff or be removed from the BFRT cuff prior to, during, and/or after use of the BFRT cuff;
a BFRT cuff that includes a battery-powered motor/pump unit;
a BFRT cuff that includes a battery-powered motor/pump unit wherein the battery is rechargeable;
a BFRT cuff that includes a motor/pump unit having one or more displays regarding a) whether the pump is on/off, b) battery status, c) pressure level for pressure in the one or more air chambers of the BFRT cuff, d) time/date, e) whether pressure in the one of more air chambers of the BFRT cuff is at or below limb occlusion pressure, f) error notification/status, g) security status (e.g., locked, unlocked, etc.), h) ambient temperature, i) timer/clock information, j) alarm status/notification, k) time of use, l) pressure mode, operation mode, and/or selected training session, m) whether full occlusion has been obtained, and/or n) whether maximum workout time period has occurred;
a BFRT cuff that includes a motor/pump unit that can be manually controlled;
a BFRT cuff that includes a motor/pump unit wherein the air pressure in the one or more air chambers can be manually controlled on the motor/pump unit itself;
a BFRT cuff that includes a pressure sensor and one or more processors to provide pressure information on the housing of the motor/pump unit itself or on some other region of the BFRT cuff;
a BFRT cuff that can be manually or automatically operated;
a BFRT cuff that allows for manual input of a predetermined limb occlusion pressure and uses such information to obtain a desired pressure in the one or more air chambers of the BFRT cuff;
a BFRT cuff that includes a sensor to automatically shut off the pump and/or release pressure in the one or more air chambers of the BFRT cuff after sensing that an occlusion pressure in the one or more air chambers has been maintained for some preset period of time (e.g., at least 30 seconds, at least 1 minute, at least 4 minutes, at least 5 minutes, etc.);
a BFRT cuff that includes a sensor to automatically shut off the pump and/or releases pressure in the one or more air chambers of the BFRT cuff after some preset period of time (e.g., 20 minutes, etc.); and/or
a BFRT cuff that includes a sensor to automatically shut off the pump and/or releases pressure in the one or more air chambers of the BFRT cuff after some preset period of time of non-use of the BFRT cuff (e.g., 10 minutes, etc.).

In another and/or alternative non-limiting aspect of the present disclosure, there is provided a BFRT cuff that includes one or more air bladders that can be inflated and deflated and a detachable air pump that is configured to inflated and deflate the one or more air bladders of the BFRT cuff. The BFRT cuff is generally formed of a flexible material (e.g., nylon material, neoprene, Kevlar™, etc.) that is used to partially or fully house the one or more air bladders. The one or more air bladders can be formed of a rubber material or any other material that can be inflated and retain air within the air bladder until deflated. The length of the BFRT cuff is generally 20-70 cm for use about an arm and generally 30-100 cm for use about a leg; however, other lengths can be used. The width of the BFRT cuff is at least 5 cm, typically about 5-14 cm, and more typically about 8-12 cm; however; other widths can be used. The BFRT cuff includes one or more air connection arrangements that are used to inflate/deflate the one or more air bladders. Generally, the one or more air connection arrangements are configured to releasably connected to a pump or air tube that is used to inflate/deflate the one or more air bladders. In one non-limiting arrangement, the one or more air connection arrangements are a quick-connect arrangement that is known in the art. The BFRT cuff generally includes one or more valves that are used to maintain and/or release air from the one or more air bladders. In one non-limiting arrangement, the air connection arrangements include one or more valves.

In another and/or alternative non-limiting aspect of the present disclosure, there is provided a portable electric pump system that is used to inflate and/or deflate the one or more air bladders in the BFRT cuff. The portable electric pump system has a housing that has a size that is less than 400 cubic inches, typically less than 300 cubic inches, and more typically less than 100 cubic inches. The weight of the housing and all of the components that are contained in the housing of the portable electric pump system is less than 40 ounces, typically less than 20 ounces, and more typically less than 16 ounces. In one non-limiting embodiment, the housing of the portable electric pump system includes a rechargeable battery. The rechargeable battery can be charged wirelessly and/or via a charge cable. The housing can optionally include a charging port for the rechargeable battery. The portable electric pump system can include an air tube that is connected at one end to the housing of the portable electric pump system and is configured to be connected (e.g., releasably connected, non-releasably connected, etc.) at the other end to an air connection arrangement on the BFRT cuff.

In another and/or alternative non-limiting aspect of the present disclosure, the portable electric pump system includes a sensor that is used to detect the air pressure in the one or more air bladders of the BFRT cuff. The portable electric pump system typically includes electronics (e.g., processor, software code, logic circuits, etc.) that are used to facilitate in the measurement calculations for pressure.

In another and/or alternative non-limiting aspect of the present disclosure, the housing of the portable electric pump system includes a display and one or more user interface buttons. The one or more buttons can include a) a power button, b) one or more arrow or scroll up/down buttons, and/or c) reset button. The display can be a LED display or the like, and may or may not be a touch screen display. Information on the display can include, but is not limited to, a) battery power level, b) pressure reading, c) warning indicator for too high of pressure, d) warning indicator for battery level too low, e) information about BFRT, f) arm or leg selection, g) limb occlusion pressure (LOP), h) day and/or time, i) time BFRT cuff pressurized, j) pressure reset, k) system reset, l) deflate BFRT cuff, m) inflate BFRT cuff, and/or n) percent of LOP to which BFRT cuff is to be pressurized, etc.

In another and/or alternative non-limiting aspect of the present disclosure, the portable electric pump system is configured to 1) pressurize the BFRT cuff until a LOP pressure is reached or a maximum preset pressure (e.g., 400 mmHg, etc.) is reached, 2) reduce the pressure in the BFRT cuff to a set percent of LOP (% LOP) (preset % LOP or manually set % LOP, and 3) then terminate operation of the air pump after the set % LOP is obtained or after a maximum preset pressure has been obtained.

In another and/or alternative non-limiting aspect of the present disclosure, the BFRT cuff can have a urethane-coated air bladder. The urethane coating on the bladder can be used to add to user comfort by reducing pinching of the skin by the bladder when inflated. The urethane coating can also optionally be used to fill in the folds that are created by the bladder when inflated.

In another and/or alternative non-limiting aspect of the present disclosure, one non-limiting method of operation of the portable electric pump system and BFRT cuff is as follows: a) removably placing a BFRT cuff (e.g., using a hook and loop strap fastener, etc.) onto a limb of a user (e.g., arm, leg, etc.), b) attach the pump or pump air hose/tube to the BFRT cuff if not already connected, c) turning on portable electric pump system if not already on, d) optionally allow or cause the portable electric pump system to calibrate to ambient pressure, e) optionally select limb (arm, leg, etc.) on the portable electric pump system (e.g., select limb listed on display of housing of the portable electric pump system, etc.), f) select % LOP (e.g., using display and one or more selection keys on housing of the portable electric pump system, the user selects a desired % LOP or a default % LOP can be selected by the user), which % LOP is generally 20%-80% and all values therebetween of the LOP, g) cause air pump to be activated to pressurize BFRT cuff until the LOP on the limb is detected, h) cause air pump and/or portable electric pump system to reduce pressure in the BFRT cuff until the selected % LOP is detected in the BFRT cuff, i) terminate operation of the air pump after the % LOP in the BFRT cuff is obtained, and j) optionally disconnect the air pump or the air hose/tube from the BFRT cuff. The % LOP is obtained in the BFRT cuff by the portable electric pump system first pressurizing the BFRT cuff until the LOP of the limb is detected and thereafter removing or allowing air to be removed from the BFRT cuff until the % LOP is obtained. The air pump typically will immediately stop inflating the BFRT cuff once LOP in the limb is detected and will thereafter cause the deflation of the BFRT cuff to the selected % LOP. For example, if LOP is detected at a pressure of 200 mmHg, then 50% LOP would be 100 mmHg and 75% LOP would be 150 mmHg. After the user completes use of the BFRT cuff when inflated to the selected % LOP, the user can a) manually partially or fully deflate the BFRT cuff and thereafter remove the BFRT cuff from the limb, or b) have the portable electric pump system partially or fully deflate the BFRT cuff and thereafter remove the BFRT cuff from the limb. If the portable electric pump system is not connected to the BFRT cuff, the portable electric pump system is first connected to the BFRT cuff prior to having the portable electric pump system partially or fully deflate the BFRT cuff.

One non-limiting object of the present disclosure is the provision of a method for exercising using a blood flow restriction training (BFRT) cuff comprising: (a) providing a BFRT cuff, said BFRT cuff formed of a flexible outer material, an air chamber that is at least partially encapsulated by said flexible outer material, an air input port that is in fluid communication with said air chamber, and a connection strap configured to secure said BFRT cuff to a limb of a user, at least a portion of said air input port positioned on an exterior surface of said BFRT cuff, said air input port including an air valve, said flexible outer material having a length of at least 20 cm and a width of least 5 cm; (b) providing a portable electric pump system, said portable electric pump system configured to at least partially inflate said BFRT cuff, said portable electric pump system includes a housing and an air connection arrangement that fluidly connects said housing to said BFRT cuff via said air input port so as to enable pumped air from said portable electric pump system to flow to and at least partially inflate said BFRT cuff, said housing has a size that is less than 400 cubic inches and has a weight that is less than 40 ounces, said housing at least partially contains i) an electric air pump, ii) a power source, and iii) a pressure sensor; (c) placing said BFRT cuff on a limb of a user; (d) connecting said BFRT cuff to said portable electric pump system to enable air flow between said BFRT cuff said portable electric pump system; (e) selecting a pressure value or percent of LOP, said pressure value less than said LOP, said percent no more than 90%; (f) causing said portable electric pump system to supply air to said BFRT cuff to thereby cause said BFRT cuff to inflate until said LOP of said limb is obtained; (g) terminating air flow from said portable electric pump system to said BFRT cuff after said LOP has been obtained for the limb; (h) causing said portable electric pump system to begin reducing pressure in said BFRT cuff within 10 seconds after said LOP is obtained in said BFRT cuff and continue to reduce pressure in said BFRT cuff until said pressure value that was selected or said percent of said LOP that was selected is obtained in said BFRT cuff; (i) disconnecting said BFRT cuff from said portable electric pump system so that said portable electric pump system cannot supply air to said BFRT cuff; and, (j) exercising the limb of said user while said BFRT cuff is at the selected pressure or the selected percent of said LOP.

Another and/or alternative non-limiting object of the present disclosure is the provision of a method for exercising using a BFRT cuff that further including the steps of: (k) reconnecting said BFRT cuff to said portable electric pump system to enable air flow between said BFRT cuff said portable electric pump system after the user has completed use of said BFRT cuff on the limb at said pressure value that was selected or said percent of said LOP that was selected; and, (l) causing said portable electric pump system to remove air from said BFRT cuff to reduce pressure in said BFRT cuff below said pressure value that was selected or said percent of said LOP that was selected.

Another and/or alternative non-limiting object of the present disclosure is the provision of a method for exercising using a blood flow restriction training (BFRT) cuff that further including the step of removing said BFRT cuff from said limb after said step of exercising the limb of said user.

Another and/or alternative non-limiting object of the present disclosure is the provision of a method for exercising using a blood flow restriction training (BFRT) cuff wherein said housing of said portable electric pump system includes a circuit board, a processor, a display, a power button, and one or more selection or scroll buttons, said one or more selection or scroll buttons configured to enable manual operation and/or control of said portable electric pump system, said display displays three or more types of information selected from the group consisting of a) measured pressure, b) power or charge level of said power source, c) a warning symbol when some type of warning event has occurred, c) selected % LOP, d) selected pressure, e) instructional information as to how to use said portable electric pump system and/or BFRT cuff, and f) arm or leg selection options for BFRT cuff.

Another and/or alternative non-limiting object of the present disclosure is the provision of a method for exercising using a BFRT cuff that further includes the step of turning on or powering up said portable electric pump system, said portable electric pump system configured to calibrate with ambient pressure after said portable electric pump system is turned on or powered up.

Another and/or alternative non-limiting object of the present disclosure is the provision of a method for exercising using a BFRT cuff that further includes the step of selecting the limb upon which said BFRT cuff is to be place.

Other objects, advantages, and novel features of the present disclosure will become apparent from the following description of the disclosure when considered in conjunction with the accompanying drawings.

### BRIEF DESCRIPTION OF DRAWINGS

FIG. 1 illustrates a BFRT cuff and a portable electric pump system in accordance with the present disclosure.

FIG. 2 illustrates a top view of a non-limiting display on the housing of the portable electric pump system.

FIG. 3 is a front view of a non-limiting housing of the portable electric pump system.

FIGS. 4-12 are illustrations of flow diagrams for the operation of the electric pump system and BFRT cuff in accordance with one non-limiting aspect of the present disclosure.

### DETAILED DESCRIPTION

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 inches to 10 inches” is inclusive of the endpoints, 2 inches and 10 inches, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

In accordance with the present disclosure, there is provided a BFRT cuff 100 for use in BFRT. The BFRT cuff includes an inflatable air system formed of one or more two layers of material. On one non-limiting embodiment, the inflatable air system includes two layers of material that are coupled together to create one or more air chambers to be inflated with air and/or some other gas. In one non-limiting configuration, the BFRT cuff 100 is a single air chamber inflatable cuff. The configuration and shape of the air chambers is non-limiting. The inflatable air system is configured in the BFRT cuff 100 such that inflation of the inflatable air system produces compression on a target compression zone that in turn produces a restriction of blood flow in the venous system of a user. The BFRT cuff 100 includes an outer material cover that has a cavity for containing the inflatable air system to isolate the inflatable air system from the skin of the user. The inflatable air system can optionally be secured in the cavity to one or more inner surface of the outer material cover (e.g., stitching, adhesive, melted connection, etc.). The outer material cover is generally made of a flexible and durable material (e.g., nylon, Kevlar™, etc.). In one non-limiting embodiment, the outer material cover is formed of an inelastic or non-stretch material. The outer material cover is configured to a) facilitate in distributing the force that is applied by the inflatable air system to the limb of a user, b) reduce pinching of the user's skin during the inflation of the inflatable air system, and/or c) provide friction between the BFRT cuff 100 and the user's skin to inhibit or prevent rotation of the BFRT cuff 100 on the user during use.

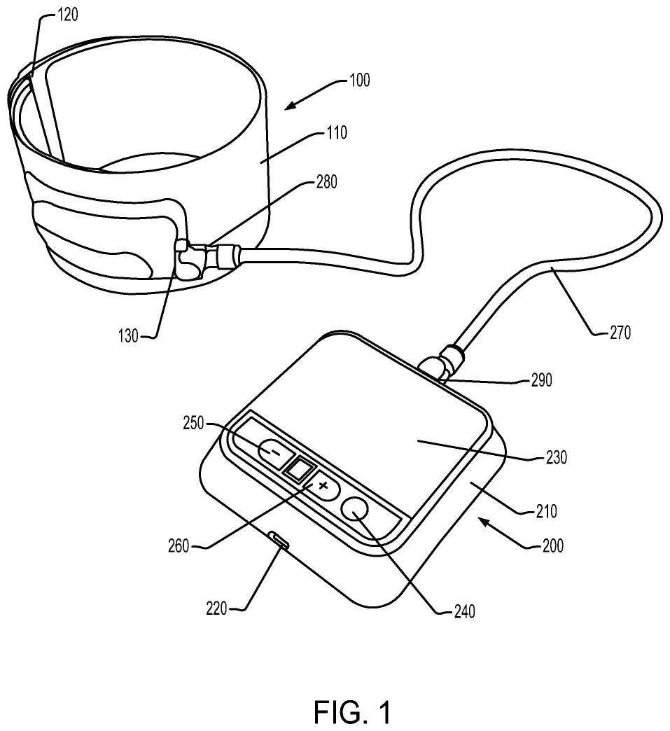

The BFRT cuff 100 generally includes a connection arrangement to facilitate in the securing of the BFRT cuff 100 to a limb of a user. As illustrated in FIG. 1, the BFRT cuff 100 includes a loop/loop like material 110 on the outer surface that is configured to releasably connect to a hook/loop material on a strap 120.

The BFRT cuff 100 includes one or more air input ports 130 that are in fluid communication with the inflatable air system to allow a fluid (e.g., air) to flow into and out of the inflatable air system. The air input port 130 generally includes a valve component. The location of the one or more air input ports 130 on the BFRT is non-limiting. Generally, at least one air input port 130 is located on an outwardly facing surface of the outer material cover. One or more of the air input ports 130 is configured to be connected to a portable electric pump system 200. The one or more air input ports 130 can optionally include a one-way valve (not shown) to prevent undesired deflation of the air inflatable system. The one or more air input ports can optionally be configured to allow for manual deflation of the air inflatable system.

As illustrated in FIG. 1, the BFRT cuff 100 can be used with a portable electric pump system 200 to inflate/deflate the BFRT cuff. The portable electric pump system 200 includes a housing 210 that at least partially contains a) an electric air pump (not shown), b) a power source (not shown), c) a pressure sensor (not shown), d) circuit board and other electronics (not shown), e) processor (not shown), f) software (not shown), g) power port/data port 220 (e.g., micro USB port, etc.), h) one or more displays 230 (e.g., LED screen, OLED screen, etc.), i) optional GPS system (not shown), j) optional wireless electronics to send/receive wireless signals (not shown), k) power button 240, l) one or more selection or scroll buttons 250, 260 to enable manual operation/control of the portable electric pump system, m) air hose port 290, and/or n) optional lights and/or other indicators (not shown) to indicate a mode and/or operation and/or status of the portable electric pump system 200. The shape and size of the housing 210 is non-limiting. The material used to form the housing 210 is generally a durable material (e.g., plastic, metal, composite, etc.).

As illustrated in FIG. 1, a first end of an air hose 270 is connected to the air hose port 290 on housing 210. The air hose 270 can be permanently connected or detachably connected to the housing 210. The second end of air hose 280 includes a hose connector 280 that is configured to releasably connect to air input port 130 on BFRT cuff 100. Generally, the type of connection between hose connector 280 and air input port 130 is a quick-connect connection arrangement; however, this is not required.

As illustrated in FIGS. 1 and 3, the front face of housing 210 includes a power port/data port 220. The type of connection is non-limiting. As can be appreciated, the power port/data port 220 can be located on other regions of housing 220. The power port/data port 220 can be used to recharge the battery in the housing 210. The power port/data port 220 can optionally be used to 1) provide software updates to the portable electric pump system 200 and/or allow for repair/reset of the portable electric pump system 200, 2) obtain diagnostic information from the portable electric pump system 200, and/or 3) download/upload user information and/or BFRT use information.

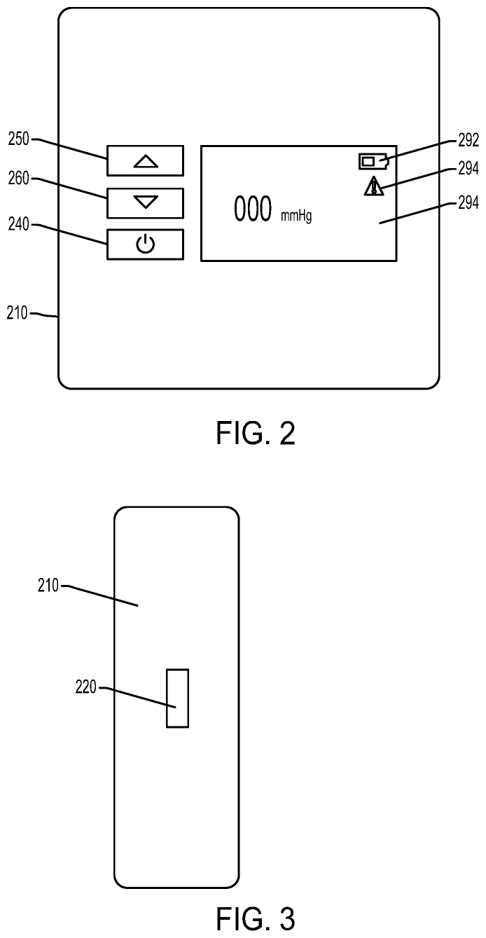

As illustrated in FIGS. 1 and 2, two types of button and display configurations are illustrated. As can be appreciated, other types of button and display configurations can be used on the housing 210 of the portable electric pump system 200. As illustrated in FIG. 1, the top of the housing 210 includes a large display 230 that covers about 50-80% (and all values and ranges therebetween) of the top surface of the housing 210. The display 230 can be a LED display or the like. Positioned below the display 230 is a power button 220 and two arrow or scroll buttons 250, 260. The power button is used to turn the portable electric pump system 200 on and off. The power button 220 can also optionally be used to reset the portable electric pump system 200. The two arrow or scroll buttons 250, 260 can be used to select items displayed on the display 230 (e.g., select % LOP, select if BFRT cuff is to be used on leg or arm, select time of exercise, select if % LOP used on one limb is to be also used on another limb, select to deflate BFRT cuff, select to inflate BFRT cuff, select to reset portable electric pump system 200, select day, select time, select to remove/reset warning on display, select to perform diagnostics and/or update on portable electric pump system 200, select to cause BFRT cuff to be pressurized to LOP for a user limb and then to depressurize BFRT cuff to selected or programmed % LOP, etc.). As can be appreciated, the arrow or scroll buttons 250, 260 can be used for other or additional functions.

As illustrated in FIG. 3, the top of the housing 210 includes a display 230, a power button 240 and two arrow or scroll buttons 250, 260; however, the orientation of the display 230 and two arrow or scroll buttons 250, 260 on the top of the housing is different. The size of the display in FIG. 3 is smaller than in FIG. 1. As illustrated in FIG. 3, the size of the display covers about 12-40% (and all values and ranges therebetween) of the top surface of the housing 210. The power button 240 and the two arrow or scroll buttons 250, 260 are positioned to the left side of the display 230 as opposed to the bottom of the display 230 as illustrated in FIG. 1. The function of the power button 240 and the two arrow or scroll buttons 250, 260 illustrated in FIG. 3 can be the same or different from the function of the power button 240 and the two arrow or scroll buttons 250, 260 described above with reference to FIG. 1. As illustrated in FIG. 3, the display shows the measured pressure in mmHg, the power or charge level of the battery in the housing 210, and optionally displays a warning symbol if some type of warning event has occurred (e.g., battery power level too low, malfunction of air pump, malfunction of electronic/processor of the portable electric pump system 200, maximum measured/detected pressure exceeded, etc.). As can be appreciated, the displays 230 of FIGS. 1 and 3 can display other or additional material (e.g., selected % LOP, operational information for the portable electric pump system 200 and/or BFRT cuff 100, LOP pressure, pressure at selected % LOP, operational state or step of the portable electric pump system 200 [e.g., inflating BFRT cuff, obtaining limb LOP, deflating BFRT cuff to selected % LOP, fully deflating BFRT cuff, etc.], manual selection of % LOP, selection of arms or legs for BFRT cuff inflation, select to fully deflate BFRT cuff, time remaining to fully charge the battery, whether the air pump is on/off, etc.).

The operation of the portable electric pump system 200 and BFRT cuff 200 will now be described:

Step 1—Turning on or powering up portable electric pump system 200 if not already turned on. The turning on or powering up of the portable electric pump system 200 can be accomplished by pressing the power button 240 on the housing 210. If the portable electric pump system 200 has been turned off or powered down, the portable electric pump system 200 can optionally be configured to calibrate with the ambient pressure when the portable electric pump system 200 is tuned on or powered up. Generally, the portable electric pump system 200 turning on or powering up of the portable electric pump system 200 will cause the display 230 to be turned on.

Step 2—Charge battery in housing 210 of the portable electric pump system 200 if not already sufficiently charged. Generally, the battery in the housing 210 should be charged at least 30-50% of a full charge prior to operation of the portable electric pump system 200 to inflate the BFRT cuff 100. The battery charge level 292 can be optionally displayed on display 230 of housing 210. In one non-limiting embodiment, the battery in the housing 210 should be charged at least 50-100% of a full charge prior to operation of the portable electric pump system 200 to inflate the BFRT cuff 100. The portable electric pump system 200 can optionally be configured to cause a warning 294 to be displayed on the display 230 of housing 210 when 1) the battery charge level is too low, and/or 2) occlusion has been detected when occlusion was not being tested thereby causing an auto-vent protocol to engage. As can be appreciated, the portable electric pump system 200 can be configured such that other or alternative events can cause the warning 294 to be displayed on display 230. The portable electric pump system 200 can optionally be configured to prevent operation of the air pump when the battery charge level is too low.

Step 3—Optionally select the limb (e.g., arm or leg) upon which the BFRT cuff 100 is to be placed. The portable electric pump system 200 can be optionally programmed such that display 230 on housing 210 requests the user to select using one or both of the two arrow or scroll buttons 250, 260 or by touching the display (if the display is a touch screen display) on which limb that the BFRT cuff 100 is to be attached during the inflation of the BFRT cuff 100. This step and feature of the portable electric pump system 200 is optional.

Step 4—Place the BFRT cuff 100 on the desired limb of the user. The step of placing the BFRT cuff 100 on the desired limb of the user can occur at any time prior to the portable electric pump system 200 causing the BFRT cuff to be inflated. During the step of placing the BFRT cuff 100 on the desired limb of the user, the proper size BFRT 100 cuff is selected (generally there are different BFRT cuff sizes for the arms and legs), and then the BFRT cuff 100 is fitted about the limb of the user, and the connection strap (e.g., hook and loop fastener strap, etc.) can be used to removably secure the BFRT cuff 100 to the limb.

Step 5—Select % LOP. The portable electric pump system 200 is configured to allow the user to select the desired % LOP to be used during the exercise of the limb. The % LOP can be selected by the user using one or both of the two arrow or scroll buttons 250, 260 or by touching the display (if the display is a touch screen display). The portable electric pump system 200 can be configured to not permit a selected % LOP greater than 90%. In one non-limiting embodiment, the portable electric pump system 200 allows the user to only select a % LOP from 20-85% and all values and ranges therebetween. The portable electric pump system 200 can optionally provide a suggested % LOP based on the input by the user of the limb upon which the BFRT cuff 100 is to be connected. The portable electric pump system 200 can optionally have preprogrammed maximum and/or minimum % LOP values so that a user is optionally unable to selected a % LOP that is greater than the maximum preprogrammed % LOP value and/or the user is optionally unable to selected a % LOP that is less than the minimum preprogrammed % LOP value.

Step 6—Connect the portable electric pump system 200 to the BFRT cuff 100 prior to activating the pump of the portable electric pump system 200. The step of connecting the portable electric pump system 200 to the BFRT cuff 100 can occur at any time prior to the portable electric pump system 200 causing the BFRT cuff 100 to be inflated. As illustrated in FIG. 1, the portable electric pump system 200 can be attached to the BFRT cuff 100 by the connection of the air tube 270 to air input port 130 of the BFRT cuff 100. As can be appreciated, the portable electric pump system 200 can be connected to the BFRT cuff 100 by other or additional arrangements.

Step 7—Cause air pump of the portable electric pump system 200 to activate to cause BFRT cuff to be inflated to the LOP of the limb upon which the BFRT cuff 100 is attached. The portable electric pump system 200 can be caused to be activated by the user using one or both of the two arrow or scroll buttons 250, 260 or by touching the display (if the display is a touch screen display), or automatically after the % LOP or selected pressure is selected by the user. The portable electric pump system 200 can optionally be configured to allow the electric air pump to continue to pump air into the BFRT cuff 100 until a maximum preset air pressure is reached in the BFRT cuff 100. The portable electric pump system 200 can optionally be configured to stop the operation of the electric air pump once the maximum preset air pressure is reached in the BFRT cuff 100, and thereafter optionally cause the BFRT cuff 100 to be partially or fully deflated. Such a feature can be used to reduce or prevent damage to the BFRT cuff 100 and/or reduce or prevent harm to the user. The portable electric pump system 200 is configured to terminate the flow of air to the BFRT cuff 100 once the LOP in the limb has been detected. The LOP can be measured by use of the oscillometric method by inflating up the BFRT cuff until it senses full occlusion (no arterial pulse) then taking an array average/mean pressure. The portable electric pump system 200 can be configured to sense full occlusion as fast as a half a millisecond such that the portable electric pump system 200 can take an average pressure in the span of full occlusion. Thereafter, the portable electric pump system 200 is configured to allow or cause air to be removed from the BFRT cuff 100 until the selected % LOP is reached in the BFRT cuff 100. Generally, the portable electric pump system 200 is configured to allow or cause air to be removed from the BFRT cuff 100 in no more than 10 seconds of the portable electric pump system 200 determining or sensing that the LOP has been obtained, and typically the portable electric pump system 200 is configured to allow or cause air to be removed from the BFRT cuff 100 in no more than 5 seconds of the portable electric pump system 200 determining or sensing that the LOP has been obtained, and more typically the portable electric pump system 200 is configured to allow or cause air to be removed from the BFRT cuff 100 in more than 2 seconds of the portable electric pump system 200 determining or sensing that the LOP has been obtained

The removal of air from the BFRT cuff 100 can be by use of a valve in the portable electric pump system 200 that is activated to allow release of air that is flowing from the BFRT cuff 100 through the air tube 270 and to the portable electric pump system 200, and/or the air pump in the portable electric pump system 200 can be configured to draw air from the BFRT cuff 100 via the air tube 270.

Once the % LOP or selected pressure has been obtained in the BFRT cuff 100, the air pump terminates operation (if operating) and/or the air release valve in the portable electric pump system 200 closes so that no further air is allowed to escape from the BFRT cuff 100 after % LOP or selected pressure has been obtained in the BFRT cuff 100. The portable electric pump system 200 can optionally be configured to provide a visual signal via the display 230 on the housing 210 and/or provide an audible sound once the user selected % LOP or selected pressure in the BFRT cuff 100 has been obtained.

Step 8—Detach the portable electric pump system 200 from the BFRT cuff 100 after the % LOP has been obtained in the BFRT cuff 100. The detachment of the portable electric pump system 200 from the BFRT cuff 100 can be accomplished by disconnecting the hose connector 280 of air hose 270 from the air input port 130 of the BFRT cuff 100. As can be appreciated, the portable electric pump system 200 can be disconnected from the BFRT cuff 100 in other manners. The disconnecting of the portable electric pump system 200 from the BFRT cuff 100 results in the portable electric pump system 200 not regulating the pressure in the BFRT cuff while the user exercises a limb while the BFRT cuff 100 is connected and partially inflated on the limb.

Step 9—Deflate the BFRT cuff 100. The partial or full deflation of the BFRT cuff 100 can be accomplished by 1) opening an air release valve on the BFRT cuff 100, 2) reconnecting the portable electric pump system 200 to the BFRT cuff 100 and then causing the portable electric pump system 200 to cause air to be removed from the BFRT cuff 100 (e.g., activating the electric air pump to cause the electric air pump to draw air from the BFRT cuff 100, and through the air tube 270 and then out from the housing 210, causing a valve in the portable electric pump system 200 to open to allow air to flow from the BFRT cuff 100, through the air tube 270 and out through the open valve, etc.).

Step 10—Remove the BFRT cuff 100 from the user's limb. After the user has completed the exercises for the limb using the BFRT cuff, the user can remove the BFRT cuff from the limb after the BFRT cuff 100 has been partially or fully deflated.

The above steps can be modified if the user wishes to perform multiple exercises of the limb using different % LOP. For example, if a user after an exercise wants to continue exercise of the limb but using a different LOP, then after Step 9, the user repeats one or more or Steps 1-3 and 5-9.

The portable electric pump system 200 can optionally be configured to allow a user to select the same % LOP or pressure used on one limb for use on another limb. For example, if a user uses the BFRT cuff 100 on his right arm and selected a % LOP of 50% (which was a pressure of 190 mmHg), the portable electric pump system 200 can optionally be configured to allow the user to use the same BFRT cuff pressure when the BFRT cuff 100 is connected to the left arm without having the portable electric pump system 200 having to determine the LOP of the left arm. In such an arrangement, the portable electric pump system 200 will merely cause the BFRT cuff 100 on the left arm to inflate to the same pressure as was used on the right arm. When such a configuration is available on the portable electric pump system 200, such option will typically be displayed on the display 230 of housing 210.

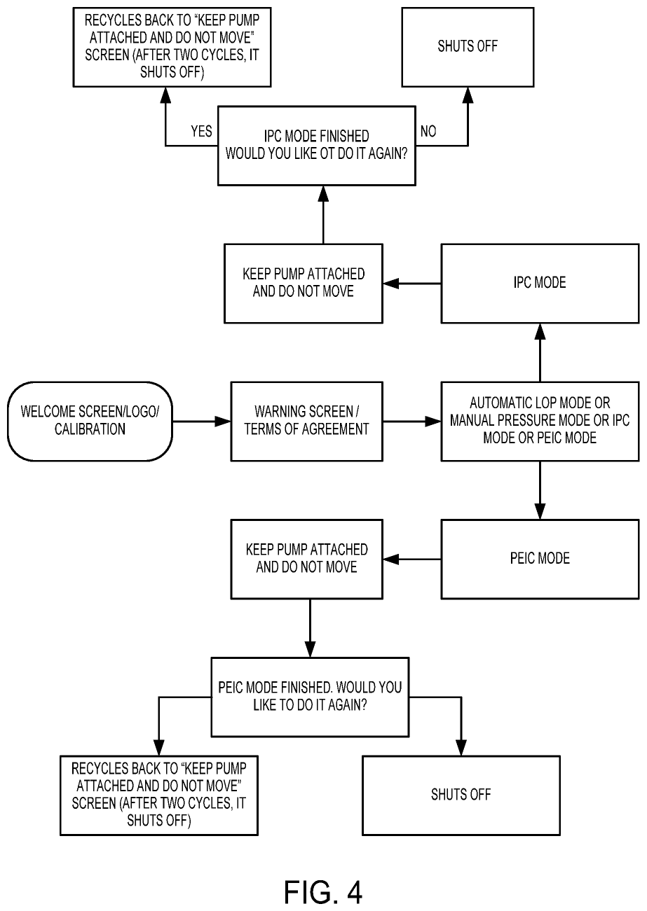

Referring now to FIGS. 4-12, there are illustrations of flow diagrams for the operation of the electric pump system 200 and BFRT cuff 100 in accordance with one non-limiting aspect of the present disclosure. FIG. 4 illustrates that the portable electric pump system 200 can operate in either the Automatic LOP mode or the Manual Pressure Mode. As it can be appreciated, the portable electric pump system 200 can be configured to only operate in the Automatic LOP mode or only operate in the Manual Pressure Mode.

Referring again to FIG. 4, after the power button 240 is pressed on housing 210 of the portable electric pump system 200, an optional Welcome Screen is displayed on display 230. Also, while the Welcome Screen is displayed on display 230, the portable electric pump system 200 can optionally be configured to conduct an ambient air pressure calibration with the one or more pressure sensors in the housing 210 of the portable electric pump system 200.

After the optional Welcome Screen, an optional Warning Screen and/or Terms Of Use Screen can be displayed. One or more buttons 250, 260 may optionally be required to be pressed or the display screen 230 touched (when display is a touch screen display) to indicate acknowledgement of the warnings and/or Terms of Use.

After the optional Warning Screen and/or Terms Of Use Screen, if the portable electric pump system 200 has more than one mode, then the two or more modes of operation of the portable electric pump system 200 can be displayed so that the user can select the mode of operation by use of the one or more buttons 250, 260 or by touching the display screen 230 (when display is a touch screen display). As illustrated in FIG. 4, the portable electric pump system 200 has four (4) operational modes (e.g., Automatic LOP mode, Manual Pressure mode, IPC mode, PEIC mode). FIG. 1 illustrates the operation of the BFRT cuff 100 and the electric pump system 200 when the IPC mode (Ischemic Preconditioning mode) or PEIC mode (Post Exercise Ischemic Conditioning mode) are selected by the user.

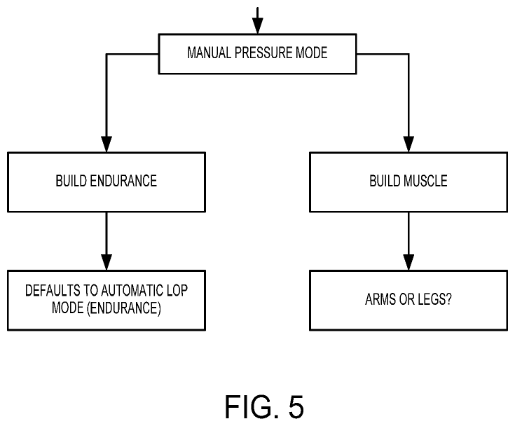

When the Manual Pressure mode is selected by the user, the operation of the BFRT cuff 100 and the electric pump system 200 will follow the operation illustrated in FIGS. 5-7. When the electric pump system 200 only has a single Manual Pressure Mode or the electric pump system 200 does not have an Automatic LOP mode and the IPC or PEIC modes are not selected, the operation of the BFRT cuff 100 and the electric pump system 200 can optionally be configured to follow the operation illustrated in FIGS. 5-7 without having the user select another mode.

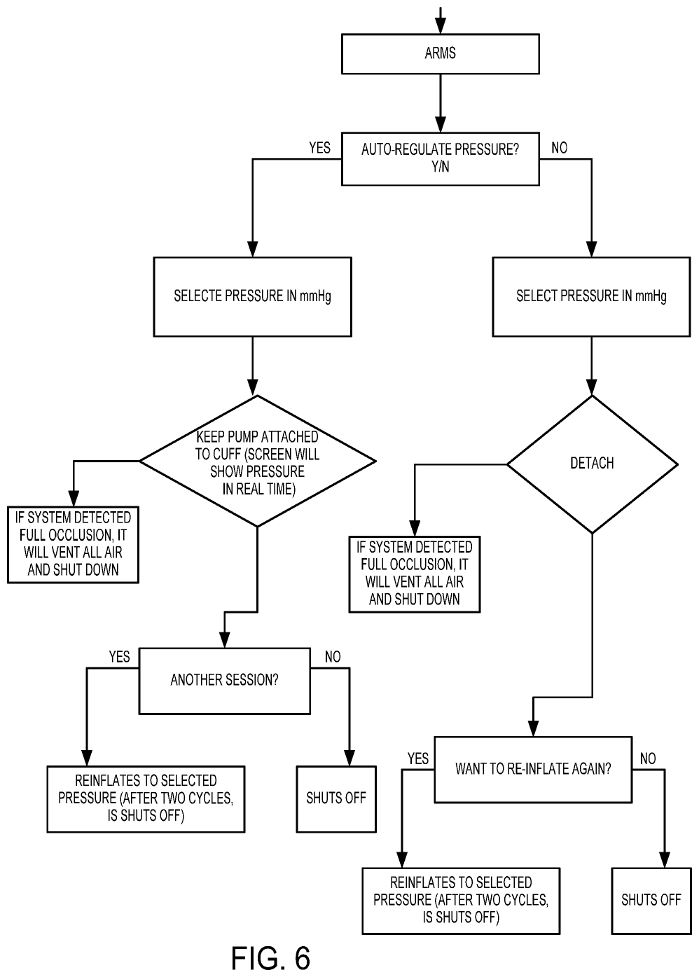

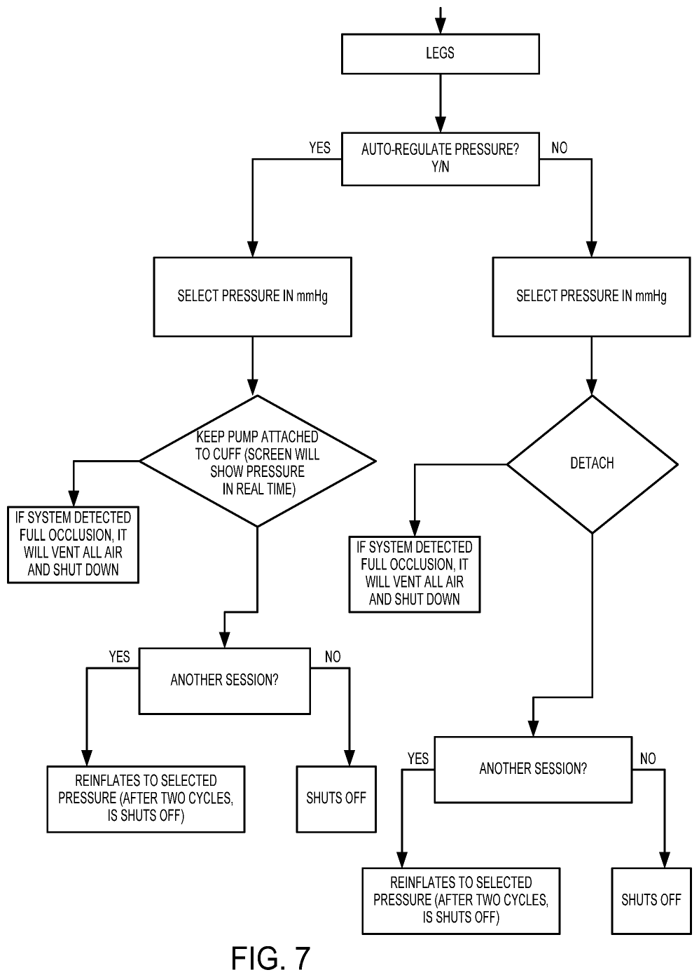

FIG. 5 illustrates that the user can select between building muscle or building endurance. However, if the BFRT cuff 100 and the electric pump system 200 does not have an Automatic LOP mode, then only the build muscle option can be used by the user. When the build muscle option is selected, the user is optionally asked to select whether the arm or leg is to have the BFRT cuff attached thereto. FIG. 6 illustrates the operation of the BFRT cuff 100 and the electric pump system 200 when the arm is selected, and FIG. 7 illustrates the operation of the BFRT cuff 100 and the electric pump system 200 when the leg is selected.

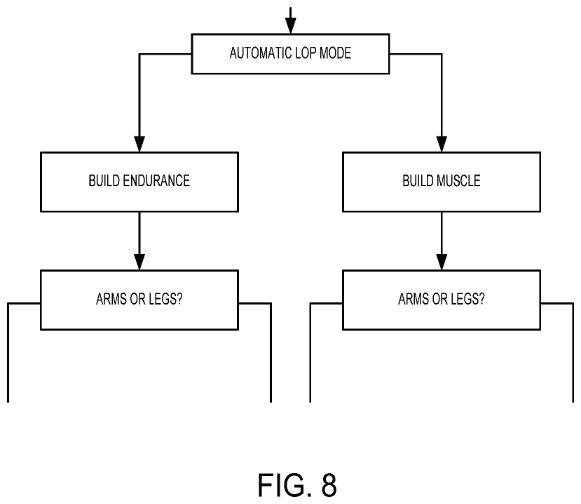

When the BFRT cuff 100 and the electric pump system 200 have an Automatic LOP mode, FIGS. 8-12 illustrated the operation of the BFRT cuff 100 and the electric pump system 200 is such mode.

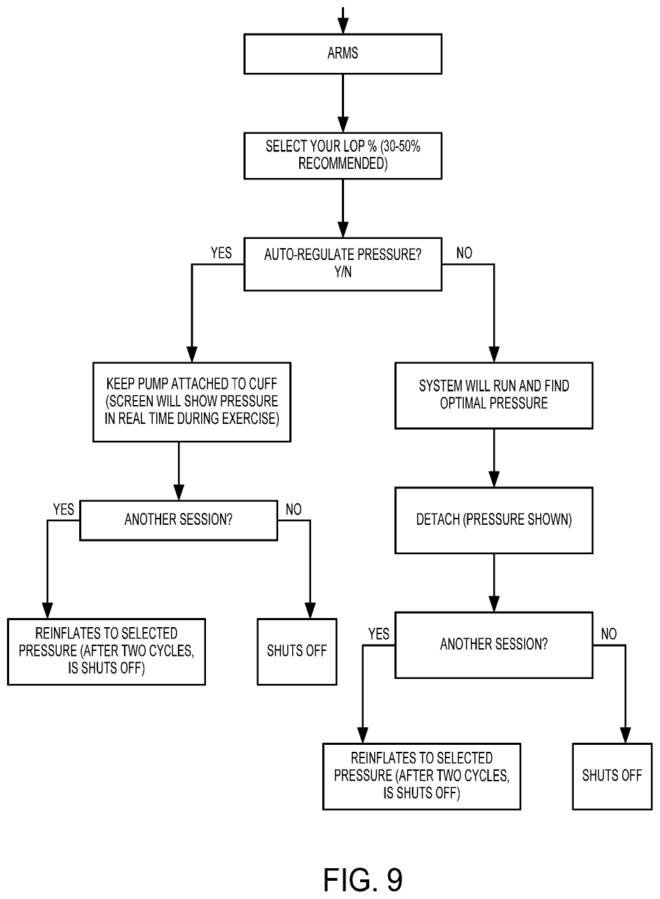

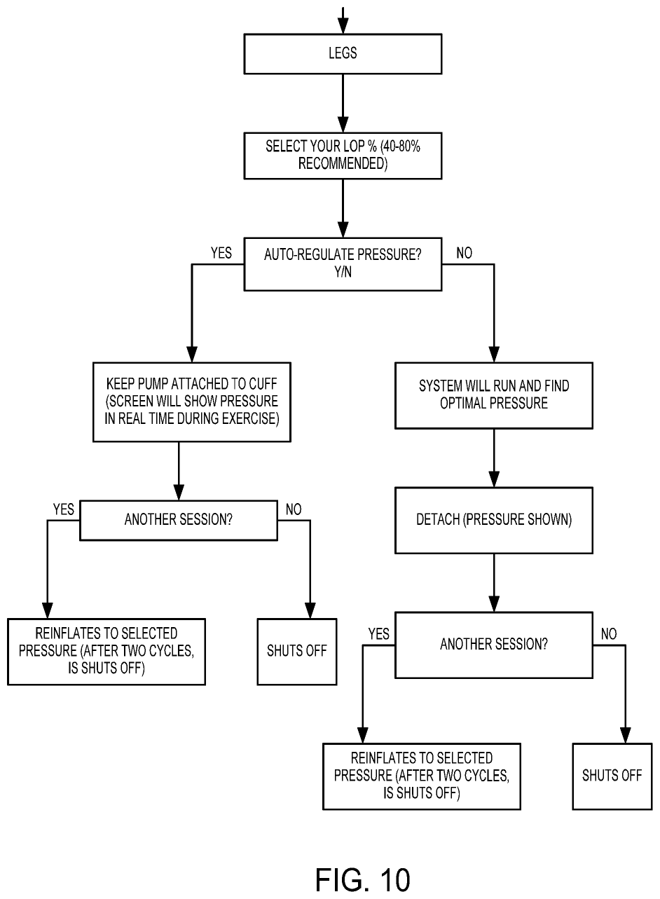

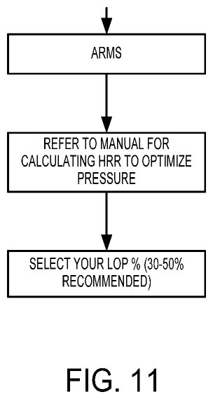

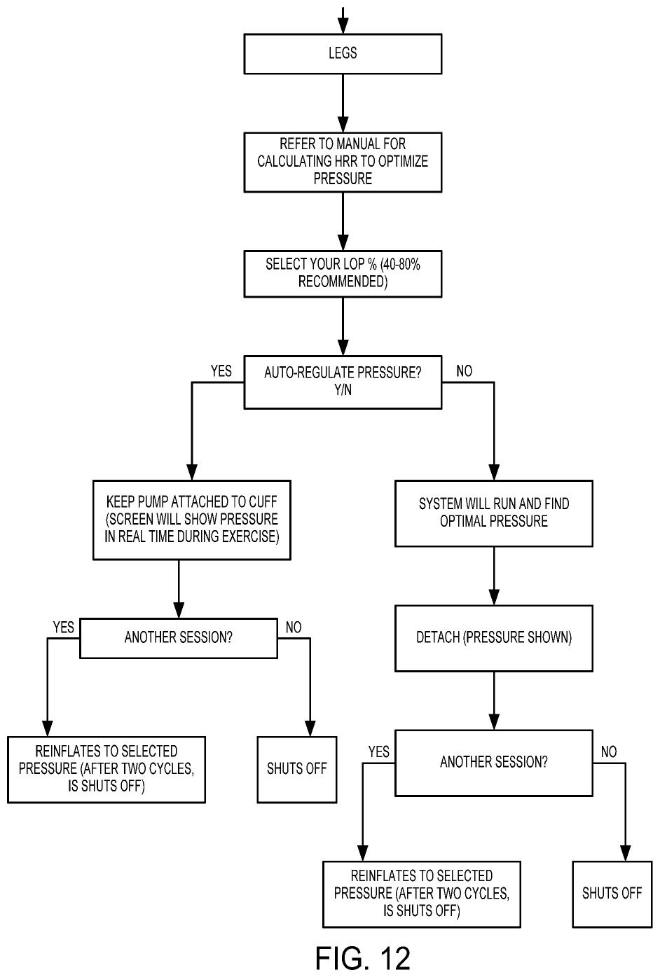

FIG. 8 illustrates that the user can optionally be asked to select the build muscle mode or the build endurance mode. After either mode is selected, the user is then optionally asked to select whether the arm or leg is to have the BFRT cuff attached thereto. FIG. 9 illustrates the operation of the BFRT cuff 100 and the electric pump system 200 when the arm is selected in the build muscle mode, FIG. 10 illustrates the operation of the BFRT cuff 100 and the electric pump system 200 when the leg is selected in the build muscle mode, FIG. 11 illustrates the operation of the BFRT cuff 100 and the electric pump system 200 when the arm is selected in the build endurance mode, and FIG. 12 illustrates the operation of the BFRT cuff 100 and the electric pump system 200 when the leg is selected in the build endurance mode.

To aid the Patent Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall there between. The invention has been described with reference to the preferred embodiments. These and other modifications of the preferred embodiments as well as other embodiments of the invention will be obvious from the disclosure herein, whereby the foregoing descriptive matter is to be interpreted merely as illustrative of the invention and not as a limitation. It is intended to include all such modifications and alterations insofar as they come within the scope of the appended claims.

What is claimed:

1. A method for exercising using a blood flow restriction training (BFRT) cuff comprising:
a. providing a BFRT cuff, said BFRT cuff formed of a flexible outer material, an air chamber that is at least partially encapsulated by said flexible outer material, an air input port that is in fluid communication with said air chamber, and a connection strap configured to secure said BFRT cuff to a limb of a user, at least a portion of said air input port positioned on an exterior surface of said BFRT cuff, said air input port including an air valve, said flexible outer material having a length of at least 20 cm and a width of least 5 cm;
b. providing a portable electric pump system, said portable electric pump system configured to at least partially inflate said BFRT cuff, said portable electric pump system includes a housing and an air connection arrangement that fluidly connects said housing to said BFRT cuff via said air input port to enable pumped air from said portable electric pump system to flow to and at least partially inflate said BFRT cuff, said housing has a size that is less than 400 cubic inches and has a weight that is less than 40 ounces, said housing at least partially contains i) an electric air pump, ii) a power source, and iii) a pressure sensor;
c. placing said BFRT cuff on a limb of a user;
d. connecting said BFRT cuff to said portable electric pump system to enable air flow between said BFRT cuff and said portable electric pump system;
e. selecting a pressure value or percent of limb occlusion pressure (LOP), said pressure value less than said LOP, said percent no more than 90%;
f. causing said portable electric pump system to supply air to said BFRT cuff to thereby cause said BFRT cuff to inflate until said LOP of said limb is obtained;
g. terminating air flow from said portable electric pump system to said BFRT cuff after said LOP has been obtained for the limb;
h. causing said portable electric pump system to begin reducing pressure in said BFRT cuff within 10 seconds after said LOP is obtained in said BFRT cuff and continue to reduce pressure in said BFRT cuff until said pressure value that was selected or said percent of said LOP that was selected is obtained in said BFRT cuff;
i. disconnecting said BFRT cuff from said portable electric pump system so that said portable electric pump system cannot supply air to said BFRT cuff; and,
j. exercising the limb of said user while said BFRT cuff is at the selected pressure or the selected percent of said LOP.

2. The method as defined in claim 1, further including the steps of:
k. reconnecting said BFRT cuff to said portable electric pump system to enable air flow between said BFRT cuff said portable electric pump system after the user has completed use of said BFRT cuff on the limb at said pressure value that was selected or said percent of said LOP that was selected; and,
l. causing said portable electric pump system to remove air from said BFRT cuff to reduce pressure in said BFRT cuff below said pressure value that was selected or said percent of said LOP that was selected.

3. The method as defined in claim 1, further including the step of removing said BFRT cuff from said limb after said step of exercising the limb of said user.

4. The method as defined in claim 2, further including the step of removing said BFRT cuff from said limb after said step of exercising the limb of said user

5. The method as defined in claim 1, wherein said housing of said portable electric pump system includes a circuit board, a processor, a display, a power button, and one or more selection or scroll buttons, said one or more selection or scroll buttons configured to enable manual operation and/or control of said portable electric pump system, said display displays three or more types of information selected from the group consisting of a) measured pressure, b) power or charge level of said power source, c) a warning symbol when some type of warning event has occurred, c) selected % LOP, d) selected pressure, e) instructional information as to how to use said portable electric pump system and/or BFRT cuff, and f) arm or leg selection options for BFRT cuff.

6. The method as defined in claim 4, wherein said housing of said portable electric pump system includes a circuit board, a processor, a display, a power button, and one or more selection or scroll buttons, said one or more selection or scroll buttons configured to enable manual operation and/or control of said portable electric pump system, said display displays three or more types of information selected from the group consisting of a) measured pressure, b) power or charge level of said power source, c) a warning symbol when some type of warning event has occurred, c) selected % LOP, d) selected pressure, e) instructional information as to how to use said portable electric pump system and/or BFRT cuff, and f) arm or leg selection options for BFRT cuff.

7. The method as defined in claim 1, further including the step of turning on or powering up said portable electric pump system, said portable electric pump system configured to calibrate with ambient pressure after said portable electric pump system is turned on or powered up.

8. The method as defined in claim 6, further including the step of turning on or powering up said portable electric pump system, said portable electric pump system configured to calibrate with ambient pressure after said portable electric pump system is turned on or powered up.

9. The method as defined in claim 1, further including the step of selecting the limb upon which said portable electric pump system that said BFRT cuff is to be place.

10. The method as defined in claim 8, further including the step of selecting the limb upon which said portable electric pump system that said BFRT cuff is to be place.
